(19) United States
(12) Patent Application Publication
(10) Pub. No.: US 2024/0377640 A1
(43) Pub. Date: Nov. 14, 2024

(54) STEREOSCOPIC DISPLAY AND DIGITAL LOUPE FOR AUGMENTED-REALITY NEAR-EYE DISPLAY

(71) Applicant: AUGMEDICS LTD., Yokneam Illit (IL)

(72) Inventors: Asaf Asaban, Haifa (IL); Yaacov Hillel Rothschild, Kiryat Yearim (IL); Nissan Elimelech, Beerotaim (IL); Stuart Wolf, Yokneam (IL)

(21) Appl. No.: 18/683,676

(22) PCT Filed: Aug. 18, 2022
(86) PCT No.: PCT/IB2022/057735

§ 371 Date: Feb. 14, 2024

Related U.S. Application Data
(60) Provisional application No. 63/281,677, filed on Nov. 21, 2021.
(60) Provisional application No. 63/236,244, filed on Aug. 24, 2021.
(60) Provisional application No. 63/236,241, filed on Aug. 24, 2021.
(60) Provisional application No. 63/234,272, filed on Aug. 18, 2021.

(51) Int. Cl. G02B 27/01; A61B 90/00; G06T 7/521; G06T 7/593; G06T 19/00; H04N 13/128; H04N 13/156; H04N 13/167; H04N 13/239; H04N 13/344; H04N 13/383; H04N 23/11

(52) U.S. Cl. CPC G02B 27/0172; A61B 90/37; G06T 7/521; G06T 7/593; G06T 19/006; H04N 13/128; H04N 13/156; H04N 13/167; H04N 13/239; H04N 13/344; H04N 13/383; H04N 23/11; A61B 2090/365; A61B 2090/367; A61B 2090/371; A61B 2090/373; G02B 2027/0134; G02B 2027/0138; G02B 2027/014; G02B 2027/0141; G02B 2027/0178; G06T 2207/10012; G06T 2207/10028; G06T 2207/30012; G06T 2210/41

(57) ABSTRACT

Disclosed herein are implementations for a digital stereoscopic display and digital loupes utilizing the digital stereoscopic display. The stereoscopic display may include display of magnified images.

### INCORPORATION BY REFERENCE TO ANY PRIORITY APPLICATIONS

This application claims the benefit of U.S. Provisional Patent Application No. 63/234,272, filed Aug. 18, 2021; U.S. Provisional Patent Application No. 63/236,241, filed Aug. 24, 2021; U.S. Provisional Patent Application No. 63/281,677, filed Nov. 21, 2021; and U.S. Provisional Patent Application No. 63/236,244, filed Aug. 24, 2021; the entire content of each of which is incorporated herein by reference.

### FIELD

The present disclosure relates generally to augmented reality, and specifically to systems and methods for use of augmented-reality near-eye displays in image-guided surgery.

### BACKGROUND

Medical practitioners use optical loupes to see a magnified image of a region of interest (ROI) during surgery and in other medical procedures. Traditionally, such optical loupes comprise magnifying optics, with fixed or variable magnification. A loupe may be, for example, integrated in a spectacle lens or may be movably mounted on a spectacle frame or on the user's head.

Near-eye display devices and systems can be used in augmented reality systems.

See-through displays (e.g., displays including at least a portion which is see-through) are used in augmented reality systems, for example for performing image-guided and/or computer-assisted surgery. Applicant's own work has demonstrated that such see-through displays can be presented as near-eye displays, e.g., integrated in a Head Mounted Device (HMD). In this way, a computer-generated image may be presented to a healthcare professional who is performing the procedure, and, in some cases, such that the image is aligned with an anatomical portion of a patient who is undergoing the procedure. Systems for image-guided surgery are described, for example, in U.S. Pat. Nos. 9,928,629, 10,835,296, 10,939,977, PCT International Publication WO 2019/211741, U.S. Patent Application Publication 2020/0163723, and PCT International Publication WO 2022/053923. The disclosures of all these patents and publications are incorporated herein by reference.

### SUMMARY

Embodiments of the present disclosure provide systems and methods for presenting augmented-reality near-eye displays. The systems and methods of the disclosure each have several innovative aspects, no single one of which is solely responsible for the desirable attributes disclosed herein.

In accordance with several embodiments, a head-mounted display device (HMD) includes a see-through display, a plurality of video cameras configured to simultaneously capture an image including a region of interest (ROI) within a predefined field of view (FOV), and a distance sensor configured to measure the distance from the HMD to the ROI. The head-mounted display device also includes at least one processor configured to determine the distance from each of the video cameras to the ROI based on the measured distance from the HMD to the ROI, and adjust the display of each image of the images captured by the video cameras on the see-through display based on the determined distances from the video cameras to provide an improved display on the see-through display.

In some embodiments, the plurality of video cameras includes two video cameras positioned symmetrically about a longitudinal plane of a wearer of the head-mounted unit such that the plurality of video cameras include a left video camera and a right video camera. Each of the left and right video cameras may include a sensor.

In some embodiments, the FOV is predefined for each of the left and right video cameras by determining a crop region on each sensor. In some embodiments, the crop regions of the sensors of the left and right video cameras are determined such that the left and right video cameras converge at a preselected distance from the HMD. In some embodiments, the crop regions of the sensors of the left and right video cameras are determined such that images captured by the left and right video cameras at a preselected distance from the HMD fully overlap.

In some embodiments, the distance sensor includes an infrared camera.

In some embodiments, the left and right video cameras each include a red-green-blue (RGB) video camera.

In some embodiments, the HMD is in the form of eyewear (e.g., goggles, glasses, visor). In some embodiments, the HMD is in the form of a helmet or over-the-head mounted device.

In some embodiments, the at least one processor is further configured to discard non-overlapping portions of the images. In some embodiments, the at least one processor is further configured to display only the overlapping portions of the images on the see-through display.

In some embodiments, the at least one processor is further configured to determine focus values corresponding to the determined distances and, for each determined distance, apply the corresponding focus value to the left and right video cameras.

In some embodiments, the at least one processor is further configured to determine a magnification value and to magnify the displayed images on the see-through display by the magnification value.

In some embodiments, the at least one processor is further configured to overlay augmented reality images on the magnified images displayed on the see-through display. The at least one processor may be further configured to magnify the overlaid augmented reality images on the see-through display by the magnification value.

In some embodiments, the augmented reality images include a 3D model of a portion of an anatomy of a patient generated from one or more pre-operative or intraoperative medical images of the portion of the anatomy of the patient (e.g., a portion of a spine of the patient, a portion of a knee of the patient, a portion of a leg or arm of the patient, a portion of a brane or cranium of the patient, a portion of a torso of the patient, a portion of a hip of the patient, a portion of a foot of the patient).

In some embodiments, the adjustment is a horizontal shift based on a horizontal shift value corresponding to the determined distances of the plurality of video cameras from the ROI.

In some embodiments, the left and right video cameras are disposed on a plane substantially parallel to a coronal plane and are positioned symmetrically with respect to a longitudinal plane. The coronal plane and the longitudinal plane may be defined with respect to a user wearing the HMD.

In some embodiments, the at least one processor is configured to determine horizontal shift values corresponding to the determined distance from the left video camera and from the right video camera to the ROI, and horizontally shift the display of each image of the images captured by the left and right video cameras on the see-through display by the corresponding horizontal shift value.

In some embodiments, the see-through display includes a left see through display and a right see-through display that are together configured to provide a stereoscopic display.

In accordance with several embodiments, a method of providing an improved stereoscopic display on a see-through display of a head-mounted display device includes simultaneously capturing images on a left and a right video camera of the head-mounted display device. The images include a region of interest (ROI) within a field of view (FOV), such as a predefined FOV. The method further includes measuring a distance from the HMD to the ROI using a distance sensor mounted on or in the head-mounted display device. The method also includes determining a distance from each of the left and right video cameras to the ROI based on the measured distance from the HMD to the ROI. The method further includes adjusting the display of each image of the images captured by the left and right video cameras on the see-through display of the head-mounted display device based on the determined distances from the left and right video cameras to provide the improved stereoscopic display on the see-through display.

The see-through display may include a left see-through display and a right see-through display. Each of the left and right video cameras may include a sensor. In some embodiments, the FOV is predefined for each of the left and right video cameras by determining a crop region on each sensor. In some embodiments, the crop regions of the sensors of the left and right video cameras are determined such that the left and right video cameras converge at a preselected distance from the HMD. In some embodiments, the crop regions of the sensors of the left and right video cameras are determined such that the images captured by the left and right video cameras at a preselected distance from the HMD fully overlap.

The distance sensor may include an infrared camera. The distance sensor may include a light source. The left and right video cameras may be red-green-blue (RGB) color video cameras.

The method may also include discarding overlapping portions of the images. The method may include displaying only the overlapping portions of the images on the see-through display.

In some embodiments, the method includes determining focus values corresponding to the determined distances and, for each determined distance, applying the corresponding focus value to the left and right video cameras.

In some embodiments, the method includes determining a magnification value and magnifying the displayed images on the see-through display by the magnification value.

In some embodiments, the method includes overlaying augmented reality images on the magnified images displayed on the see-through display. The method may also include magnifying the overlaid augmented reality images on the see-through display by the magnification value.

In some embodiments, the adjusting includes applying a horizontal shift based on a horizontal shift value corresponding to the determined distances of the left and right video cameras from the ROI.

The methods may be performed by one or more processors within the head-mounted display device or communicatively coupled to the head-mounted display device.

In accordance with several embodiments, an imaging apparatus for facilitating a medical procedure, such as a spinal surgery, includes a head-mounted unit including a see-through display and at least one video camera, which is configured to capture images of a field of view (FOV), having a first angular extent, that is viewed through the display by a user wearing the head-mounted unit and a processor configured to process the captured images so as to generate and present on the see-through display a magnified image of a region of interest (ROI) having a second angular extent within the FOV that is less than the first angular extent.

In some embodiments, the head-mounted unit comprises an eye tracker configured to identify a location of a pupil of an eye of the user wearing the head-mounted unit. In some embodiments, the processor is configured to generate the magnified image responsively to the location of the pupil. In some embodiments, the eye tracker is configured to identify respective locations of pupils of both a left eye and a right eye of the user. In some embodiments, the processor may be configured to measure an interpupillary distance responsively to the identified locations of the pupils via the eye tracker and to present respective left and right magnified images of the ROI on the see-through display responsively to the interpupillary distance.

In some embodiments, the magnified image presented by the processor comprises a stereoscopic image of the ROI. The at least one video camera may include left and right video cameras, which are mounted respectively in proximity to left and right eyes of the user. The processor may be configured to generate the stereoscopic image based on the images captured by both the left and right video cameras.

In some embodiments, the processor is configured to estimate a distance from the head-mounted unit to the ROI based on a disparity between the images captured by both the left and right video cameras, and to adjust the stereoscopic image responsively to the disparity.

In some embodiments, the see-through display includes left and right near-eye displays. The processor may be configured to generate the stereoscopic image by presenting respective left and right magnified images of the ROI on the left and right near-eye displays, while applying a horizontal shift to the left and right magnified images based on a distance from the head-mounted unit to the ROI.

In some embodiments, the head-mounted unit includes a tracking system configured to measure the distance from the head-mounted unit to the ROI. In some embodiments, the tracking system includes a distance sensor. The distance sensor may include an infrared camera.

In some embodiments, the processor is configured to measure the distance by identifying a point of contact between a tool held by the user and the ROI.

In some embodiments, the FOV comprises a part of a body of a patient undergoing a surgical procedure (e.g., an open surgical procedure or a minimally invasive interventional procedure).

In some embodiments, the processor is configured to overlay an augmented reality image on the magnified image of the ROI that is presented on the see-through display.

In accordance with several embodiments, a method for imaging includes capturing images of a field of view (FOV), having a first angular extent, using at least one video camera mounted on a head-mounted unit, which includes a see-through display through which a user wearing the head-mounted unit views the FOV. The method also includes processing the captured images so as to generate and present on the see-through display a magnified image of a region of interest (ROI) having a second angular extent within the FOV that is less than the first angular extent.

In some embodiments, the method includes identifying a location of a pupil of an eye of the user wearing the head-mounted unit, wherein processing the captured images comprises generating the magnified image responsively to the location of the pupil. In some embodiments, identifying the location includes identifying respective locations of pupils of both a left eye and a right eye of the user and measuring an interpupillary distance responsively to the identified locations of the pupils. In some embodiments, generating the magnified image comprises presenting respective left and right magnified images of the ROI on the see-through display with a horizontal shift applied to the left and right magnified images.

In some embodiments, the magnified image presented on the see-through display comprises a stereoscopic image of the ROI.

In some embodiments, capturing the images includes capturing left and right video images using left and right video cameras, respectively, mounted respectively in proximity to left and right eyes of the user, and processing the captured images comprises generating the stereoscopic image based on the images captured by both the left and right video cameras.

In some embodiments, the method includes estimating a distance from the head-mounted unit to the ROI based on a disparity between the images captured by both the left and right video cameras and adjusting the stereoscopic image responsively to the disparity.

In accordance with several embodiments, a computer software product, for use in conjunction with a head-mounted unit, which includes a see-through display and at least one video camera, which is configured to capture images of a field of view (FOV), having a first angular extent, that is viewed through the display by a user wearing the head-mounted unit, includes: a tangible, non-transitory computer-readable medium in which program instructions are stored, which instructions, when read by a processor, cause the processor to process the captured images so as to generate and present on the see-through display a magnified image of a region of interest (ROI) having a second angular extent within the FOV that is less than the first angular extent.

For purposes of summarizing the disclosure, certain aspects, advantages, and novel features are discussed herein. It is to be understood that not necessarily all such aspects, advantages, or features will be embodied in any particular embodiment of the disclosure, and an artisan would recognize from the disclosure herein a myriad of combinations of such aspects, advantages, or features.

The embodiments will be more fully understood from the following detailed description thereof, taken together with the drawings.

### BRIEF DESCRIPTION OF THE DRAWINGS

Non-limiting features of some embodiments are set forth with particularity in the claims that follow. The following drawings are for illustrative purposes only and show non-limiting embodiments. Features from different figures may be combined in several embodiments.

FIG. 1 is a schematic pictorial illustration showing an example head-mounted unit with digital loupe capabilities in use in a surgical procedure, in accordance with an embodiment of the disclosure;

FIG. 2 is a schematic pictorial illustration showing details of the head-mounted unit of FIG. 1;

FIG. 3 is a flow chart that schematically illustrates a method for generating magnified images for display;

FIG. 4 is a schematic pictorial illustration showing a magnified image presented in a portion of a display, in accordance with an embodiment of the disclosure;

FIG. 5 is a schematic figure illustrating an example head-mounted unit, according to an embodiment of the disclosure;

FIG. 6 is a flow chart that schematically illustrates a method for calibrating a stereoscopic digital loupe, in accordance with an embodiment of the disclosure; and

FIG. 7 is a flow chart that schematically illustrates a method for generating a stereoscopic digital loupe display, in accordance with an embodiment of the disclosure.

### DETAILED DESCRIPTION

Embodiments of the present disclosure that are described herein provide a digital stereoscopic display and digital loupes utilizing the digital stereoscopic display, in which the digital loupes include a head-mounted video camera and electronic display. In accordance with several embodiments, the digital stereoscopic display and digital loupes described herein advantageously offer a simple off-axis (or parallel) visible light camera setup utilizing a digital convergence and a utilization of a distance or tracking camera of a head-mounted display (HMD) device to provide one or more of the following benefits: (i) less consumption of resources, (ii) robust automatic focusing, (iii) robust stereoscopic tuning. (iv) reduced size and weight, by comparison with traditional optical loupes, and/or (v) improved versatility and ease of use in adjusting the display to accommodate, for example, the user's pupil spacing, region of interest, and/or desired magnification.

In addition, embodiments disclosed herein provide a stereoscopic display of a scene, and specifically, stereoscopic magnification of a scene, to a user (e.g., wearer of the HMD device) without or with minimal visual discomfort and/or visual fatigue. In accordance with several embodiments, such a display may be especially advantageous when displaying images of a scene which is relatively proximate, or close, to the user (e.g., distance around 0.5 meter or up to one meter from the user or wearer), such as when displaying images of a surgical site to a surgeon or other healthcare professional while he or she is operating on a patient or performing an interventional procedure. In accordance with several embodiments, digital loupes can be integrated advantageously with head-mounted displays (e.g., over-the-head mounted device displays or eyewear displays), such as displays that are used, for example, in systems for image-guided surgery, computer-assisted navigation, and stereotactic surgery.

The surgery may comprise open surgery or minimally-invasive surgery (e.g., keyhole surgery, endoscopic surgery, or catheter-based interventional procedures that do not require large incisions, such as incisions that are not self-sealing or self-healing without staples, adhesive strips, or other fasteners or adhesive elements).

Alternatively, stereoscopic display and digital loupes of this sort can be used in other medical applications to provide the practitioner with a stereoscopic and optionally magnified view for purposes of treatment and/or diagnosis.

In some implementations, the digital loupes provide a stereoscopic display that is convergence-based. A distance from the digital loupes to a region of interest may be determined using an optical tracking device or system (such as an infrared camera) or can be set manually by a user or operator. In some implementations, the digital loupes provide stereoscopic viewing during a surgical or other interventional procedure. In some implementations, the digital loupes facilitate adjustment of magnification, focus, angle or view, or other display setting adjustment based on both visible color video camera images (e.g., obtained from one or more RGB cameras) and images received from a tracking device (e.g., an infrared camera or sensor). In some implementations, a single device may be capable of color video and tracking (e.g., an RGB-IR device that includes one or more RGB cameras and one or more infrared cameras or sensors). The tracking device may be used to determine distance or depth measurements from the digital loupes to the region of interest.

In the disclosed embodiments, an imaging apparatus comprises a head-mounted unit (e.g., over-the-head unit or eyewear unit, such as glasses or goggles or visor) with a see-through display and at least one visible light camera (e.g., a video camera) which captures images of a field of view (FOV) that is viewed through the display by a user wearing the head-mounted unit. A processor (integrated within the head-mounted unit or external to the head-mounted unit) processes the captured images so as to generate and present (e.g., output), on the see-through display, a stereoscopic and optionally magnified image of a region of interest (ROI) within the FOV. In accordance with several embodiments, the angular extent of the ROI is less than the total angular extent of the FOV. One or more algorithms may be executed by one or more processors of, or communicatively coupled to, the digital loupes for stereoscopic display of the magnified image.

Other embodiments could include a see-through display that is not head-mounted but is mounted to one or more arms or mechanical structures such that the display is hands-free and mounted over the ROI.

In some embodiments, the processor generates and presents a magnified stereoscopic image on the see-through display, so that the user is able to see a magnified 3D-like view of the ROI. The 3D-like view may be formed by generating a three-dimensional effect which adds an illusion of depth to the display of flat or two-dimensional (2D) images, e.g., images captured by the visible light cameras. The 3D-like view may include 2D or 3D images (e.g., pre-operative and/or intraoperative anatomical medical images), virtual trajectories, guides or icons, digital representations of surgical tools or instruments, operator instructions or alerts, and/or patient information). For this purpose, inter alia, the head-mounted unit (e.g., over-the-head unit or eyewear) may comprise left and right video cameras, which are mounted such that once the HMD device is worn by a user, the cameras will be located in a symmetrical manner with respect to the user's (wearer's) nose. Accordingly, the left and right video cameras may be disposed on a plane substantially parallel to a coronal plane and in a symmetrical manner with respect to a longitudinal plane of users wearing the HMD device. The processor generates the stereoscopic image based on the images captured by both the left and right video cameras. For stereoscopic viewing, the display may comprise left and right near-eye displays, which present respective left and right images (e.g., non-magnified or magnified images) of the ROI in front of the user's left and right eyes, respectively. In several implementations, the processor applies a shift (e.g., horizontal shift) to the left and right magnified images based on the distance from the head-mounted unit to the ROI. The processor may estimate this distance by various distance measurement means, as described further hereinbelow.

The disclosed systems, software products and methods for stereoscopic display may generally apply to the display of images, and specifically to the display of magnified images, in which, any discrepancy between right and left eye images may have a more prominent effect on the quality of the stereoscopic display and the user's (wearer's) experience, including visual discomfort and visual fatigue. Furthermore, such discrepancies and their shortcomings may be further enhanced when the images are displayed on a near-eye display and in an augmented reality setting. The systems, software products and methods described herein will be described with respect to the display of magnified images and for generating a digital loupe, but may also apply, mutatis mutandis, to the display of non-magnified images.

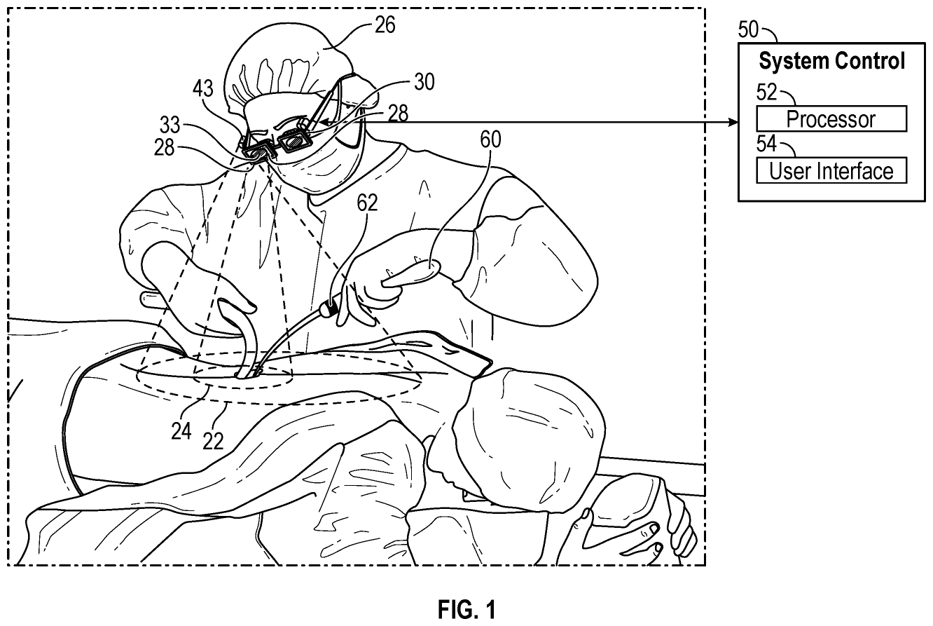

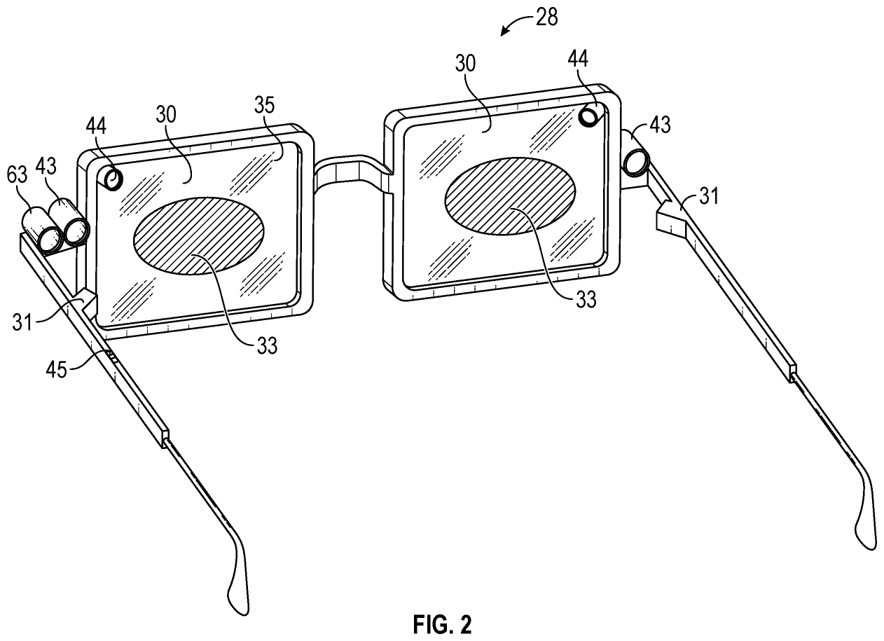

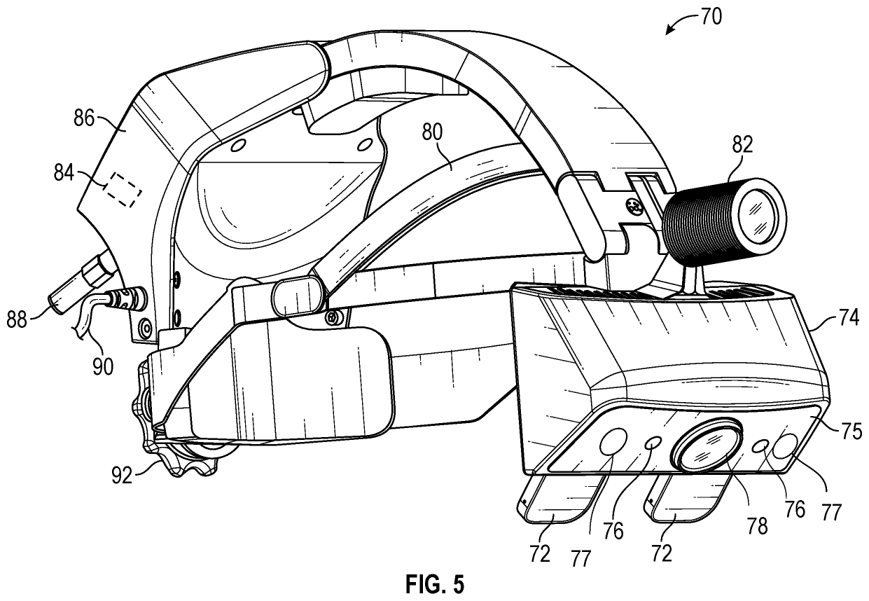

Reference is now made to FIGS. 1 and 2, which schematically illustrate a head-mounted unit 28 with digital loupe capabilities, in accordance with some embodiments of the disclosure. Head-mounted unit 28 displays magnified images of a region of interest (ROI) 24 viewed by a user, such as a healthcare professional 26. FIG. 1, for example, is a pictorial illustration of a surgical scenario in which head-mounted unit 28 may be used, while FIG. 2, for example, is a pictorial illustration showing details of an example of a head-mounted unit 28 in the form of eyewear. In some embodiments, the head-mounted unit 28 can be configured as an over-the-head mounted headset that may be used to provide digital loupe functionality such as is shown in FIG. 5 and described hereinbelow.

In the embodiment illustrated in FIG. 1, head-mounted unit 28 comprises eyewear (e.g., glasses or goggles) that includes one or more see-through displays 30, for example as described in Applicant's U.S. Pat. No. 9,928,629 or in the other patents and applications cited above, whose disclosure is incorporated herein by reference. Displays 30 may include, for example, an optical combiner, a waveguide, and/or a visor. Displays 30 may be controlled by one or more computer processors. The one or more computer processors may include, for example, a computer processor 52 disposed in a central processing system 50 and/or a dedicated computer processor 45 disposed in head-mounted unit 28. The one or more processors may share processing tasks and/or allocate processing tasks between the one or more processors. The displays 30 may be configured (e.g., programmed upon execution of stored program instructions by the one or more computer processors) to display an augmented-reality image (e.g., one or more 2D images or 3D images) to healthcare professional 26, who is wearing the head-mounted unit 28.

In some embodiments, the augmented reality image viewable through the one or more see-through displays 30 is a combination of objects visible in the real world with the computer-generated image. In some embodiments, each of the one or more see-through displays 30 comprises a first portion 33 and a second portion 35. In some embodiments, the one or more see-through displays 30 display the augmented-reality image.

According to some aspects, the augmented-reality image is presented on displays 30 such that a magnified image of ROI 24 is projected onto the first portion 33, in alignment with the anatomy of the body of the patient that is visible to healthcare professional 26 through the second portion 35. Alternatively, the magnified image may be presented in any other suitable location on displays 30, for example above the actual ROI 24 or otherwise not aligned with the actual ROI 24. Displays 30 may also be used to present additional or alternative augmented reality images (e.g., one or more 2D images or 3D images or 3D-like images), such as described in U.S. Pat. No. 9,928,629 or the other patents and applications cited above.

To capture images of ROI 24, head-mounted unit 28 includes one or more cameras 43. In some embodiments, one or more cameras 43 are located in proximity to the eyes of healthcare professional 26. Camera(s) 43 are located alongside the eyes in FIG. 2; but alternatively, camera(s) 43 may be mounted elsewhere on unit 28, for example above the eyes or below the eyes. According to some aspects, only one camera 43 may be used, e.g., mounted above the eyes near a center of the head-mounted unit 28 or at another location. Camera(s) 43 may comprise any suitable type of miniature color video cameras (e.g., RGB cameras or RGB-IR cameras), including an image sensor (e.g., CMOS sensor) and objective optics (and optionally a color array filter). In accordance with several embodiments, camera(s) 43 capture respective images of a field of view (FOV) 22, which may be considerably wider in angular extent than ROI 24, and may have higher resolution than is required by displays 30.

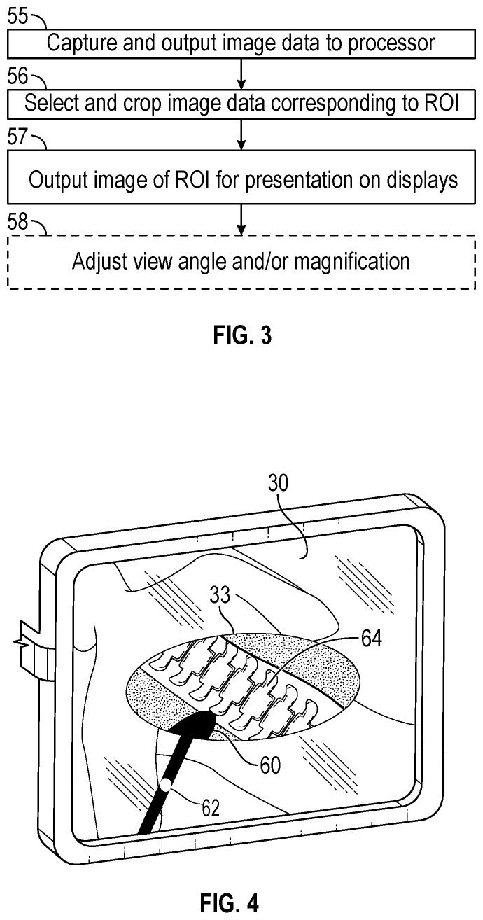

FIG. 3 is a flow chart that schematically illustrates an example method for generating magnified images for presentation on displays 30. To generate the magnified images that are presented on displays 30, camera(s) 43 (at an image capture step 55) capture and output image data with respect to FOV 22 to processor 45 and/or processor 52. At a data selection step 56, the processor 45, 52 selects and/or crops the part of the image data corresponding to ROI 24. According to some aspects, the processor 45, 52 may select and/or crop a central portion of the image. According to some aspects, the processor 45, 52 may receive or process only information received from a predefined portion, for example a predefined central portion, of the image sensor or light sensor (e.g., CMOS sensor or charge-coupled device image sensor) of the camera(s) 43. Optionally, the processor 45, 52 may then crop a further portion of this predefined portion (e.g., further reduce the information received from the image sensor or light sensor of the camera(s) 43).

In accordance with several embodiments, to improve the stereoscopic view and prevent eye discomfort, the processor 45, 52 may discard non-overlapping portions of the images captured by the left and/or right cameras 43. Non-overlapping image portions may be image portions which show portions of the FOV 22 not captured by both right and left cameras 43, but only by one of the cameras 43. Thus, in accordance with several embodiments, only an overlapping portion of the right and left images corresponding to a portion of the FOV 22 captured by both right and left cameras 43 will be displayed to the user (e.g., wearer) to generate a proper stereoscopic view.

Based on the image information received from cameras 43, the processor 45, 52 (at an image display step 57) generates and outputs a magnified image of the ROI 24 for presentation on displays 30. The magnified images presented on the left and right displays 30 may be shifted (e.g., horizontally shifted) to give healthcare professional 26 a better stereoscopic view. In some embodiments, processor 45 and/or 52 may be configured to adjust the resolution of the magnified images of the ROI 24 to match the available resolution of displays 30, so that the eyes see an image that is clear and free of artifacts. According to some aspects, healthcare professional 26 may adjust the FOV 22 (which includes ROI 24) by altering a view angle (e.g., vertical view angle to accommodate the specific user's height and/or head posture), and/or the magnification of the image that is presented on displays 30, for example by means of a user interface 54 of processing system 50 (optional user adjustment step 58). User interface 54 may comprise hardware elements, such as knobs, buttons, touchpad, touchscreen, mouse, and/or a joystick, as well as software-based on-screen controls (e.g., touchscreen graphical user interface elements and/or voice controls (e.g., voice-activated controls using a speech processing hardware and/or software module). Additionally, or alternatively, the vertical view angle of the head-up display unit may be manually adjusted by the user (e.g., via a mechanical tilt mechanism).

The head-mounted unit 28 may be calibrated according to the specific types of users or to the specific user (e.g., to accommodate the distance between the user's pupils (interpupillary distance) or to ranges of such a distance) and his or her preferences (e.g., visualization preferences). For this purpose, in some embodiments, the location of the portion of the displays 30 on which images are presented (e.g., displays portion 33 of FIG. 2), or the setup of camera(s) 43, or other features of head-mounted unit 28 may be produced and/or adjusted according to different ranges of measurements of potential users or may be custom-made, according to measurements provided by the user, such as healthcare professional 26. Alternatively or additionally, the user may manually adjust or fine-tune some or all of these features to fit his or her specific measurements or preferences.

In some embodiments, the head-mounted unit is configured to display and magnify an image, assuming the user's gaze would be typically straightforward. In some embodiments, the user's pupils' location, gaze and/or line of sight may be tracked. For example, one or more eye trackers 44 may be integrated into head-mounted unit 28, as shown in FIG. 2, for real-time adjustment and possibly for purposes of calibration. Eye trackers 44 comprise miniature video cameras, possibly integrated with a dedicated infrared light source, which capture images of the eyes of the user (e.g., wearer) of head-mounted unit 28. Processor 45 and/or 52 or a dedicated processor in eye trackers 44 processes the images of the eyes to identify the locations of the user's pupils. Additionally or alternatively, eye trackers 44 may detect the direction of the user's gaze using the pupil locations and/or by sensing the angle of reflection of light from the user's corneas.

In some embodiments, processor 45 and/or processor 52 uses the information provided by eye trackers 44 with regard to the pupil locations in generating a magnified image for presentation on displays 30. For example, the processor 45, 52 may dynamically determine a crop region on each sensor of each camera to match the user's gaze direction. Accordingly, the image generated based on the part of the sensor corresponding to the shifted crop region or ROI 24 may be magnified and output for display.

For improved stereoscopic display, the processor 45, 52 may be programmed to calculate and apply the shift (e.g., horizontal shift) to the left and right images presented on displays 30 to reduce the parallax between the user's eyes at the actual distance from head-mounted unit 28 to ROI 24. In other words, the shift (e.g., horizontal shift) of the left and right images depends on the distance and geometry of the cameras (e.g., relative to the ROI 24). The distance to the ROI 24 can be estimated by the processor 45, 52 in a number of different ways, as will be described further below:
In some embodiments, the processor 45, 52 may measure the disparity between the images of ROI 24 captured by left and right cameras 43 based on image analysis and may compute the distance to the ROI 24 based on the measured disparity and the known baseline separation between the cameras 43.
In some embodiments, based on signals provided by the one or more eye trackers 44, the processor 45, 52 may compare the gaze angles of the user's left and right eyes to find the distance at which the eyes converge on ROI 24.
In some embodiments, head-mounted unit 28 may comprise a distance sensor or tracking device 63, which measures the distance from the head-mounted unit 28 to ROI 24. The distance sensor or tracking device 63 may comprise an infrared sensor, an image-capturing tracking camera, an optical tracker, or other tracking/imaging device for determining location, orientation, and/or distance. The distance sensor or tracking device 63 may also include a light source to illuminate the ROI 24 such that light reflects from a marker on a patient or tool toward the distance sensor or tracking device 63. In some embodiments, an image-capturing device of the tracking device 63 comprises a monochrome camera with a filter that passes only light in the wavelength band of light source. In one implementation, the light source may be an infrared light source, and the camera may include a corresponding infrared filter. In other implementations, the light source may comprise any other suitable type of one or more light sources, configured to direct any suitable wavelength or band of wavelengths of light, and mounted on head-mounted unit 28 or elsewhere in the operating room.
In some embodiments, distance sensor or tracking device 63 may comprise a depth sensor configured to illuminate the FOV 22 with a pattern of structured light (e.g., via a structured light projector) and capture and process or analyze an image of the pattern on the FOV 22 in order to measure the distance. In this case, distance sensor or tracking device 63 may comprise a monochromatic pattern projector such as of a visible light color and a visible light camera.

In some embodiments, the processor 45, 52 may measure the distance from head-mounted unit 28 to an element in or adjacent to the ROI 24 while, utilizing, for example, a tracking camera of the head-mounted unit 28. In such embodiments, distance sensor 63 may be the tracking camera. With reference to FIG. 1, tool 60 may be manipulated by healthcare professional 26 within ROI 24 during the surgical or other interventional or diagnostic medical procedure. The tool 60 may be, for example, a tool used for inserting a surgical implant, such as a pedicle screw, stent, cage, or interbody device, into the body (e.g., bone, vessel, body lumen, tissue) of a patient. For this purpose, for example, tool 60 may comprise an optical marker 62 (example shown in FIG. 1), having a known pattern detectable by distance sensor or tracking device 63. An optical patient marker (not shown in the figures), which may be fixedly attached to the patient (e.g., to the patient's skin or a portion of the patient's anatomy, such as a portion of the patient's spine) may also be detectable by distance sensor or tracking device 63. The processor 45, 52 may process images of marker 62 in order to determine (e.g., measure) the location and orientation of tool 60 with respect to the head-mounted unit 28 or wearer of the head-mounted unit 28, and thus to determine (e.g., estimate or calculate) the distance between the ROI 24 and the user (e.g., wearer of the head-mounted unit 28). The distance may be determined by the distance sensor 63 (such as an infrared camera, optical sensor, or other tracking device). In some embodiments, the processor 45, 52 may process images of the patient marker or of the patient marker and tool marker in order to determine the relative location and orientation of the patient marker or of patient marker and tool marker with respect to the head mounted unit 28 or the user, and thus to determine the distance between the user and ROI 24. Such head mounted display systems are described, for example, in the above-referenced U.S. Pat. Nos. 9,928,629, 10,835,296, 10,939,977, PCT International Publication WO 2019/211741, U.S. Patent Application Publication 2020/0163723, and PCT International Publication WO 2022/053923, which were previously incorporated by reference. Markers are described, for example, in U.S. Pat. No. 10,939,977, the content of which is also hereby incorporated herein by reference.

The processor 45, 52 may compute the distance to ROI 24 based on any one of the above methods, or a combination of such methods or other methods that are known in the art. Alternatively or additionally, healthcare professional 26 may adjust the shift (e.g., horizontal shift) or location of the overlapping portions of the captured images manually.

In accordance with several embodiments, utilizing optical tracking of the head mounted unit 28 as disclosed above to dynamically provide the distance to the ROI 24 allows for a less resource consuming and more robust distance measurement, for example with respect to distance measurement based on image analysis.

The distance sensor or tracking device 63 may comprise a light source and a camera (e.g., camera 43 and/or an IR camera). The light source may be adapted to simply illuminate the ROI 24 (e.g., a projector, a flashlight or headlight). The light source may alternatively include a structured light projector to project a pattern of structured light onto the ROI 24 that is viewed through displays 30 by a user, such as healthcare professional 26, who is wearing the head-mounted unit 28. The camera (e.g., camera(s) 43 and/or infrared camera) may be configured to capture an image of the pattern on the ROI and output the resulting distance or depth data to processor 52 and/or processor 45. The distance or depth data may comprise, for example, either raw image data or disparity values indicating the distortion of the pattern due to the varying depth of the ROI 24.

Alternatively, distance sensor or tracking device 63 may apply other depth mapping technologies in generating the depth data. For example, the light source may output pulsed or time-modulated light, and the camera (e.g., camera 43) may be modified or replaced by a time-sensitive detector or detector array to measure the time of flight of the light to and from points in the ROI 24. These and all other suitable alternative depth mapping technologies are considered to be within the scope of the present disclosure.

FIG. 4 is a schematic pictorial illustration showing a magnified image presented in portion 33 of display 30, in accordance with an embodiment of the disclosure. The magnified image shows an incision 62 made by healthcare professional 26 in a back 60 of a patient, with an augmented-reality overlay 64 showing at least a portion of the patient's vertebrae (e.g., cervical vertebrae, thoracic vertebrae, lumbar vertebrae, and/or sacral vertebrae) and/or sacroiliac joints, in registration with the magnified image. For example, overlay 64 may include a 2D image or a 3D image or model of the region of interest (ROI) 24 magnified to the same proportion as the magnified image displayed in portion 33 (e.g., a video image). The overlay 64 may be then augmented or integrated, for example, on the digitally magnified video image and in alignment with the magnified image. Overlay 64 may be based, for example, on a medical image (e.g., obtained via computed tomography (CT), X-ray, or magnetic resonance imaging (MRI) systems) acquired prior to and/or during the surgical procedure or other interventional or diagnostic procedure (e.g., open surgical procedure or minimally invasive procedure involving self-sealing incisions, such as catheter-based intervention or laparoscopic or keyhole surgery). The overlay image may be aligned or otherwise integrated with the magnified image by using image analysis (e.g., by feature-based image registration techniques). In some embodiments, such alignment and/or registration may be achieved by aligning the overlay image with the underlying anatomical structure of the patient, while assuming the magnified image is substantially aligned with the patient anatomy. Alignment and/or registration of such an overlay with the underlying anatomical structure of a patient is described, for example, in the above-mentioned U.S. Pat. No. 9,928,629, which was previously incorporated by reference, and as well as in US Patent Application Publication 2021/0161614, the entire contents of which are incorporated herein by reference. In some embodiments, one or more eye trackers (e.g., eye trackers 44) may be employed which may allow a more accurate alignment of the magnified video image with the underlying patient anatomy. The eye tracker may allow capturing the ROI and in addition a display of the image on the near-eye display in alignment with the user's line of sight and the ROI in a more accurate manner when the user is not looking straightforward.

In some procedures, such as discectomy or spinal fusion, the surgeon needs to identify the patient bone structure for purposes of localization and navigation to a site of interest. The surgeon may then remove tissue and muscles to reach or expose the bone, at least to some extent. This preliminary process of “cleaning” the bone may require time and effort. The site of interest may be then magnified, for example using digital magnification, to facilitate the identification of the patient anatomy and the performance of the procedure. It may be still challenging, however, to identify the patient anatomy and navigate during the procedure due to tissue and muscles left in the site of interest.

To address this difficulty, a 3D spine model (generated from an intraoperative or preoperative CT scan or other medical image scan) can be presented with (e.g., superimposed on or integrated into) the magnified video image of the patient anatomy, as shown in FIG. 4. The alignment of this image with the patient's anatomy can be achieved by means of a registration process, which utilizes a registration marker mounted on an anchoring implement, for example a marker attached to a clamp or a pin. Registration markers of this sort are shown and described, for example, in the above-mentioned U.S. Pat. No. 9,928,629, in US Patent Application Publication 2021/0161614, which were previously incorporated by reference, as well as in US Patent Application Publication 2022/0142730, the entire contents of which are incorporated herein by reference. For this purpose, an intraoperative CT scan or other medical image scan of the ROI 24 is performed, including the registration marker. An image of the ROI 24 and of a patient marker attached (e.g., fixedly attached) to the patient anatomy or skin and serving as a fiducial for the ROI 24 is captured, for example using a tracking camera such as distance sensor or tracking device 63 of head-mounted unit 28 or camera 78 of head-mounted unit 70. The relative location and orientation of the registration marker and the patient marker are predefined or determined, e.g., via the tracking device. The CT or other medical image and tracking camera image(s) may then be registered based on the registration marker and/or the patient marker. The anatomical image model (e.g., CT model) may be then displayed in a magnified manner (corresponding to the video magnification) and aligned with the video image.

The anatomical image model (e.g., CT model) may be presented on display(s) 30, for example, in a transparent manner, in a semi-transparent manner, in an opaque manner, or in a substantially opaque manner and/or as an outline of the bone structure. Thus, in accordance with several embodiments, the surgeon or healthcare professional 26 will advantageously be able to “see” the bone structure which lies beneath tissue shown in the video image and/or “see” it in a clearer manner. This will facilitate localization and navigation (for example of tool 60) in the patient's anatomy.

Furthermore, using such a view may shorten the “cleaning” process or even render it unnecessary.

Other images may be included (e.g., augmented on or integrated with) the magnified video image, such as a planning indication (such as planning of a bone-cut or insertion of an implant, such as a bone screw or cage).

The presentation of such information in an augmented manner on the video image may be controlled by the user (e.g., on or off or presentation adjustment via the user interface 54).

Additional examples of procedures in which the above may be utilized include vertebroplasty, vertebral fusion procedures, removal of bone tumors, treating burst fractures, or when bone fracturing is required to handle a medical condition (such as scoliosis) or to access a site of interest. Other examples may include arthroscopic procedures (including joint replacement, such as hip replacement, knee replacement, shoulder joint replacement or ankle joint replacement; reconstructive surgery (e.g., hip surgery, knee surgery, ankle surgery, foot surgery); joint fusion surgery; laminectomy; osteotomy; neurologic surgery (e.g., brain surgery, spinal cord surgery, peripheral nerve procedures); ocular surgery; urologic surgery; cardiovascular surgery (e.g., heart surgery, vascular intervention); dental surgery; oncology procedures; biopsies; organ transplants; or other medical procedures.

FIG. 5 is a schematic pictorial illustration showing details of a head-mounted display (HMD) unit 70, according to another embodiment of the disclosure. HMD unit 70 may be worn by healthcare professional 26, and may be used in place of head-mounted unit 28 (FIG. 1). HMD unit 70 comprises an optics housing 74 which incorporates a camera 78, and in the specific embodiment shown, an infrared camera. In some embodiments, the housing 74 comprises an infrared-transparent window 75, and within the housing, e.g., behind the window, are mounted one or more, for example two, infrared projectors 76. Additionally or alternatively, housing 74 may contain one or more color video cameras 77, as in head-mounted unit 28, and may also contain eye trackers, such as eye trackers 44.

In some embodiments, mounted on housing 74 are a pair of augmented reality displays 72, which allow healthcare professional 26 to view entities, such as part or all of patient 24, through the displays 72, and which are also configured to present to healthcare professional 26 images or any other information. In some embodiments, displays 72 may also present stereoscopic images of ROI 24 (e.g., video images) and particularly magnification of such images of ROI 24 (FIG. 1), as described above.

In some embodiments, HMD unit 70 includes a processor 84, mounted in a processor housing 86, which operates elements of the HMD unit. In some embodiments, an antenna 88, may be used for communication, for example with processor 52 (FIG. 1).

In some embodiments, a flashlight 82 may be mounted on the front of HMD unit 70. In some embodiments, the flashlight may project visible light onto objects so that the professional is able to clearly see the objects through displays 72. In some embodiments, elements of the HMD unit 70 are powered by a battery (not shown in the figure), which supplies power to the elements via a battery cable input 90.

In some embodiments, HMD unit 70 is held in place on the head of healthcare professional 26 by a head strap 80, and the healthcare professional 26 may adjust the head strap by an adjustment knob 92.

Elements shown and described with respect to HMD unit 70, such as antenna 88 and flashlight 82, may be also included, mutatis mutandis, in HMD unit 28, and vice versa.

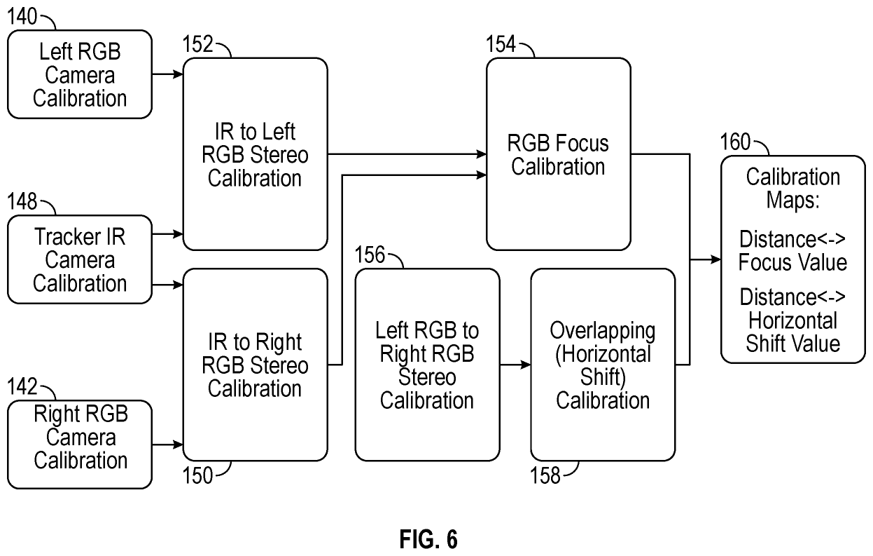

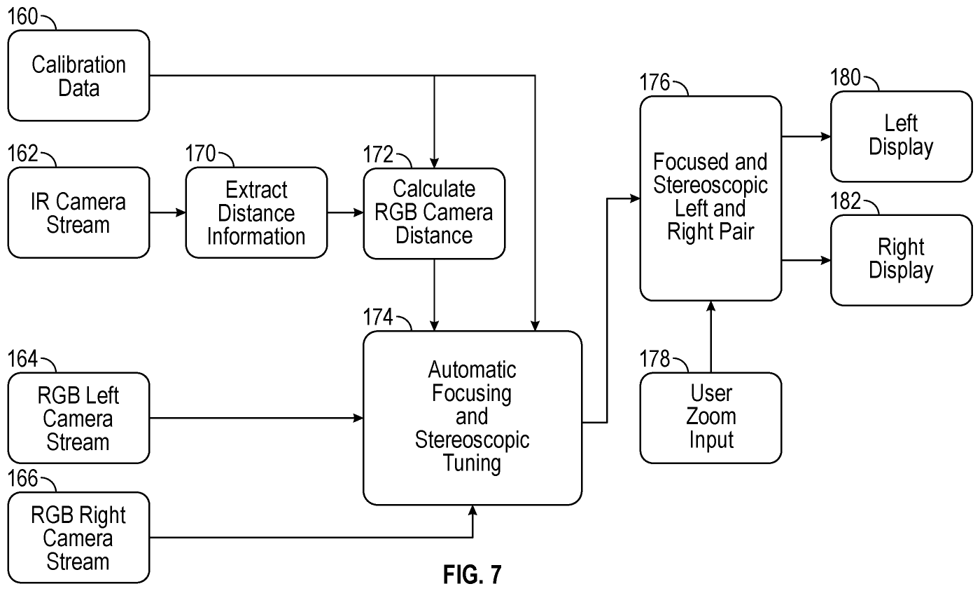

FIG. 6 is a flow chart that schematically illustrates a method for calibrating a stereoscopic digital loupe, in accordance with an embodiment of the disclosure. For the sake of clarity and concreteness, this method, as well as the method of FIG. 7, is described hereinbelow with reference to the components of head-mounted unit 28 (FIGS. 1 and 2). The principles of these methods, however, may similarly be applied to other stereoscopic digital loupes, such as a loupe implemented by HMD unit 70.

In general, in the context of the present description, when a computer processor is described as performing certain steps, these steps may be performed by one or more external computer processors (e.g., processor 52) and/or one or more computer processors (e.g., processor 45, 84) that is integrated within the HMD unit 28, 70. The processor or processors carry out the described functionality under the control of suitable software, which may be downloaded to the system in electronic form, for example over a network, and/or stored on tangible, non-transitory computer-readable media, such as electronic, magnetic, or optical memory.

In accordance with several embodiments, in generating and presenting magnified stereoscopic images, it is important that the visible light cameras be properly calibrated and registered with one another and with the tracking device. The calibration may include both one or more color video cameras and a tracking device, such as an infrared camera or sensor (e.g., distance sensor 63). In some embodiments, right and left cameras 43 (e.g., color video cameras, such as RGB cameras) and an infrared tracking camera (e.g., an infrared tracking camera in distance sensor or tracking device 63) are calibrated by one or more processors (such as processor 45, 52), at camera calibration steps 140, 142, and 148. These steps may be carried out, for example, by capturing images of a test pattern using each of the cameras and processing the images to locate the respective pixels and their corresponding 3D locations in the captured scene. If appropriate, the camera calibration may also include estimation and correction of distortion in each of the cameras. In some implementations, at least one of the right and left cameras and infrared tracking camera comprises an RGB-IR camera that includes both color video and infrared sensing or imaging capabilities in a single device.

After the individual cameras have been calibrated, the processor 45, 52 may register, by rigid transformations, the infrared camera with the right color video camera and with the left color video camera at right and left camera calibration steps 150 and 152, correspondingly. Such registration may include measuring the distances between the optical centers of each of color video cameras 43 and the infrared camera in distance sensor or tracking device 63, at right and left camera calibration steps 150 and 152. The processor 45, 52 may also measure the respective rotations of the color cameras 43 and the infrared camera of the distance sensor or tracking device 63. These calibration parameters or values serve as inputs for a focus calibration step 154, in which the focusing parameters of cameras 43 are calibrated against the actual distance to a target that is measured by the distance sensor or tracking device 63. On the basis of this calibration, it may be possible to focus both cameras 43 to the distance of ROI 24 that is indicated by the distance sensor or tracking device 63.

For enhanced accuracy in accordance with several embodiments, right and left cameras 43 (e.g., color video cameras) may also be directly registered at a stereo calibration step 156. The registration may include measurement of the distance between the optical centers of right and left cameras 43 and the relative rotation between the cameras 43, and may also include rectification, for example. At an overlapping calibration step 158, the processor 45, 52 may use these measurements in calculating an appropriate shift (e.g., horizontal shift) to be applied on the display of each of the images captured by the left and right cameras 43 (e.g., color video cameras) in correspondence to the cameras' distance from the ROI 24. The horizontal shift is applied in the display of each image and to the center of the overlapping portion of the image such that the center of the overlapping image portion is shifted to the center of the display area (e.g., to the center of portion 33 of display 30 of HMD unit 28). This application may be performed to reduce the parallax between pixels of the right and left eye images to improve the stereoscopic display, as will be further detailed in connection with FIG. 6. The overlapping image portions may vary as a function of the distance from cameras 43 to ROI 24.

At the conclusion of this calibration process, the processor 45, 52 stores calibration maps at step 160 in a memory that is associated with head-mounted unit 28. The calibration maps may include the mapping between ROI distance and the focusing parameter of cameras 43, as calculated at step 154, and a mapping between ROI distance and the horizontal shift of the overlapping left and right camera image portions that have been calculated at step 158.

The calibration maps or calibration mapping may include or refer to the generation of a lookup table, one or more formulas, functions, or models or to the estimation of such. Accordingly, processor 45, 52 may obtain or calculate the focus and/or horizontal shift values while using such one or more lookup tables, formulas, models or a combination of such once distance to the ROI is provided.

According to some embodiments, cameras 43 are mounted on the HMD unit 28 in a parallel or off-axis setup, as shown, for example in FIG. 2. To allow a stereoscopic view, at least some of the right and left images should overlap. Such overlapping may occur when the right and left cameras 43 or the right and left cameras' FOVs at least partially converge. An actual FOV of a camera may be determined, for example, by defining a crop region on the camera sensor. In a parallel setup of cameras, such overlap may not occur or may be insignificant at planes which are substantially or relatively close to the cameras (e.g., at a distance of 0.5 meter, 0.4 to 0.6 meters, or up to one meter from the user, such as when displaying images of a patient surgical site to a surgeon while he is operating on the patient).

In such a case, a digital convergence may be generated by horizontally shifting the crop region on the cameras' sensors. A crop region may be determined on the sensor of each camera such that a full overlap between the right and left images is received at a determined distance from the ROI plane. The crop regions of the right and left cameras sensors may be identical in size (to receive same image size) and symmetrical around the centers of the sensors. A digital convergence may be generated at a determined distance from the ROI by changing or horizontally shifting each of the crop regions of the cameras' sensors to an asymmetrical location with respect to the corresponding sensor center. Furthermore, the crop regions may be shifted such that a complete or full image overlapping is received at a determined distance from the ROI e.g., while the user or wearer of the head-mounted unit 28, 70 is standing at that distance, looking straightforward at the ROI and while the camera's plane is parallel to the ROI plane. A full image overlap may be received when the scene displayed by one image is identical or the same as the scene display by the other image. A full image overlap may allow the user to receive maximal information available by the configuration of the cameras (e.g., actual sensors FOV determined by the crops regions of the sensors).

Alternatively, in accordance with some embodiments, the cameras setup may not be parallel, such that a digital convergence will not be required. However, such a setup may have effects, such as vertical parallax, which may significantly reduce the quality of the stereoscopic display, in some embodiments.

In a parallel setup, a convergence and advantageously full overlap plane distance and corresponding sensor crop regions may be predetermined. Such a distance will be referred to herein as the default distance. For example, for a surgery setting, this may be the typical working distance of a surgeon 22 wearing the HMD unit 28, 70 from the surgical site or ROI 24. A full images overlap allows the user (e.g., wearer of the HMD unit 28, 70) to receive the maximal information allowed by the configuration of the cameras 43 (e.g., actual sensors FOV).

Accordingly, the calibration process as described in and with respect to FIG. 6, may include calibration such that the default or initial focus value will be a focus value corresponding to the default distance. The calibration of the cameras may be with respect to the determined sensor crop regions. The real time adjustment as described hereinbelow with reference to FIG. 7, may be performed with respect to the determined default distance and sensor crop regions.

FIG. 7 is a flow chart that schematically illustrates a method for generating a stereoscopic digital loupe display, in accordance with an embodiment of the disclosure. This method receives as its inputs a stream of infrared video images (e.g., block 162) output by distance sensor or tracking device 63 and respective streams of color video images (e.g., blocks 164, 166) that are output by the left and right color cameras 43, along with calibration data (e.g., calibration maps) generated (e.g., calculated) and stored as described in step 160 in connection with FIG. 6. Left camera and right camera may have the same frame rate, e.g., 60 frames per second. In some embodiments, left camera image capturing and/or image stream 164 and right camera image capturing and/or image stream 166 may be synchronized. In some embodiments, IR camera image capturing and/or image stream 162, left camera image capturing and/or image stream 164 and right camera image capturing and/or image stream 166 are synchronized. In a setting in which the distance between the cameras and the ROI does not change rapidly, for example, the distance between a surgeon wearing the HMD and a surgical site during a medical procedure, there may be an offset between the capturing and/or image streaming of right and left cameras. The processor 45, 52 processes infrared video images from block 162 in order to extract distance information at a distance extraction step 170. The processor 45, 52 calculates the distance from each of the cameras (e.g., from each of cameras 43) to ROI 24 based on the extracted distance from the distance sensor or tracking device 63 (e.g., infrared camera of the distance sensor or tracking device 63) to the ROI 24, e.g., using the registrations calculated at steps 150 and 152 of FIG. 6, correspondingly, at a distance calculation step 172.

The processor 45, 52 then sets the focusing parameters or values of cameras 43 to match the distance to ROI 24, based on calibration data (e.g., calibration maps) generated at block 160, at a focusing step 174. The processor 45, 52 also tunes the stereoscopic display by shifting, (e.g., horizontally shifting) the overlapping image portions and discarding the non-overlapping image portions. The processor 45, 52 applies the horizontal shift values in calibration maps or according to the mapping generated at block 160 in displaying the pair of images captured simultaneously or substantially simultaneously by right and left cameras 43 on right and left displays 30, correspondingly. The horizontal shift map or mapping values are configured such that in each distance the center of the overlapping portion in each image is shifted to the center of display portion 33 to reduce parallax and allow a better stereoscopic view and sensation. Thus, in accordance with several embodiments, the horizontal parallax between the centers of the overlapping image portions is zero or substantially zero. In some embodiments the horizontal shift value may correspond to the horizontal shift length (e.g., in pixels). In some embodiments, the horizontal shift value may correspond to the coordinates (e.g., in the display coordinate system) of the center pixel of the overlapping image portion. Furthermore, the non-overlapping image portions may be discarded. Portions of the non-overlapping image portions may be discarded simply due to the horizontal shift which places them externally to display portion(s) 33.

Consequently, these image portions may not be displayed to the user. The rest of the non-overlapping image portions or all of the non-overlapping image portions may be discarded, for example, by darkening their pixels and/or by cropping. The result of this step is a stream of focused image pairs (block 176), having only overlapping content, for proper stereoscopic presentation on displays 30. The magnification of these stereoscopic image pairs is set to a desired value, which may be optionally adjusted in accordance with a user-controlled zoom input (block 178). The resulting left and right magnified images (blocks 180, 182) are output to left and right displays 30, respectively, and are updated as new images are captured and processed.

It should be noted that the process described in FIG. 7 except for step 160 may be repeatedly performed (e.g., once in a predefined time interval and up to cameras 43 image capture rate), and such that a video of the captured ROI 24 is stereoscopically displayed on display 30. In some embodiments, the crop region of the sensors of cameras 43 may be dynamically determined or dynamically changed in real-time based on the repeatedly determined distance from cameras 43 to the ROI 24 such that for each distance a full images overlap is received. In some embodiments, the eye trackers 44 may be employed to dynamically determine the ROI 24 by dynamically and repeatedly determining the user's gaze direction or line-of-sight. The dynamic determination of the sensors crop region may then be dynamically or repeatedly determined also based on the current or simultaneously determined ROI.

The processors 45, 52 may include one or more central processing units (CPUs) or processors, which may each include a conventional or proprietary microprocessor. The processors 45, 52 may be communicatively coupled to one or more memory units, such as random-access memory (RAM) for temporary storage of information, one or more read only memory (ROM) for permanent storage of information, and one or more mass storage devices, such as a hard drive, diskette, solid state drive, or optical media storage device. The processors 45, 52 (or memory units communicatively coupled thereto) may include modules comprising program instructions or algorithm steps configured for execution by the processors 45, 52 to perform any of all of the processes or algorithms discussed herein. The processors 45, 52 may be communicatively coupled to external devices (e.g., display devices, data storage devices, databases, servers, etc. over a network via a network communications interface.

In general, the word “module,” as used herein, refers to logic embodied in hardware or firmware, or to a collection of software instructions, possibly having entry and exit points, written in a programming language, such as, for example, Java, Lua, C, C#, or C++. A software module or product may be compiled and linked into an executable program, installed in a dynamic link library, or may be written in an interpreted programming language such as, for example, BASIC, Perl, or Python. It will be appreciated that software modules may be callable from other modules or from themselves, and/or may be invoked in response to detected events or interrupts. Software modules configured for execution on computing devices may be provided on a computer readable medium, such as a compact disc, digital video disc, flash drive, or any other tangible medium. Such software code may be stored, partially or fully, on a memory device of the executing computing device, such as the processors 45, 52, for execution by the computing device. Software instructions may be embedded in firmware, such as an EPROM. It will be further appreciated that hardware modules may be comprised of connected logic units, such as gates and flip-flops, and/or may be comprised of programmable units, such as programmable gate arrays or processors. The modules described herein are preferably implemented as software modules but may be represented in hardware or firmware. Generally, any modules or programs or flowcharts described herein may refer to logical modules that may be combined with other modules or divided into sub-modules despite their physical organization or storage.

Although the drawings relate specifically to surgery on the spine, the principles of the present disclosure may similarly be applied in loupes for other sorts of medical and dental procedures, as well loupes for other applications, such as but not limited to arthroscopic procedures (including joint replacement, such as hip replacement, knee replacement, shoulder joint replacement or ankle joint replacement; reconstructive surgery (e.g., hip surgery, knee surgery, ankle surgery, foot surgery); joint fusion surgery; laminectomy; osteotomy; neurologic surgery (e.g., brain surgery, spinal cord surgery, peripheral nerve procedures); ocular surgery; urologic surgery; cardiovascular surgery (e.g., heart surgery, vascular intervention); oncology procedures; biopsies; tendon or ligament repair; and/or organ transplants.

In the foregoing specification, the systems and processes have been described with reference to specific embodiments thereof. It will, however, be evident that various modifications and changes may be made thereto without departing from the broader spirit and scope of the embodiments disclosed herein. The specification and drawings are, accordingly, to be regarded in an illustrative rather than restrictive sense.

Indeed, although the systems and processes have been disclosed in the context of certain embodiments and examples, it will be understood by those skilled in the art that the various embodiments of the systems and processes extend beyond the specifically disclosed embodiments to other alternative embodiments and/or uses of the systems and processes and obvious modifications and equivalents thereof. In addition, while several variations of the embodiments of the systems and processes have been shown and described in detail, other modifications, which are within the scope of this disclosure, will be readily apparent to those of skill in the art based upon this disclosure. It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes of the embodiments of the disclosed systems and processes. Any methods disclosed herein need not be performed in the order recited. Thus, it is intended that the scope of the systems and processes herein disclosed should not be limited by the particular embodiments described above.

It will be appreciated that the systems and methods of the disclosure each have several innovative aspects, no single one of which is solely responsible or required for the desirable attributes disclosed herein. The various features and processes described above may be used independently of one another or may be combined in various ways. All possible combinations and sub-combinations are intended to fall within the scope of this disclosure.

Certain features that are described in this specification in the context of separate embodiments also may be implemented in combination in a single embodiment. Conversely, various features that are described in the context of a single embodiment also may be implemented in multiple embodiments separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination. No single feature or group of features is necessary or indispensable to each and every embodiment.

Each of the processes, methods, and algorithms described in the preceding sections may be embodied in, and fully or partially automated by, code modules executed by one or more computer systems or computer processors comprising computer hardware. The code modules may be stored on any type of non-transitory computer-readable medium or computer storage device, such as hard drives, solid state memory, optical disc, and/or the like. The systems and modules may also be transmitted as generated data signals (for example, as part of a carrier wave or other analog or digital propagated signal) on a variety of computer-readable transmission mediums, including wireless-based and wired/cable-based mediums, and may take a variety of forms (for example, as part of a single or multiplexed analog signal, or as multiple discrete digital packets or frames). The processes and algorithms may be implemented partially or wholly in application-specific circuitry. The results of the disclosed processes and process steps may be stored, persistently or otherwise, in any type of non-transitory computer storage such as, for example, volatile or non-volatile storage.

The various features and processes described above may be used independently of one another or may be combined in various ways. All possible combinations and sub-combinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The example systems and components described herein may be configured differently than described. For example, elements may be added to, removed from, or rearranged compared to the disclosed example embodiments.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

The terms “comprising,” “including,” “having,” and the like are synonymous and are used inclusively, in an open-ended fashion, and do not exclude additional elements, features, acts, operations, and so forth. In addition, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise. Similarly, while operations may be depicted in the drawings in a particular order, it is to be recognized that such operations need not be performed in the particular order shown or in sequential order, or that all illustrated operations be performed, to achieve desirable results. Further, the drawings may schematically depict one or more example processes in the form of a flowchart. However, other operations that are not depicted may be incorporated in the example methods and processes that are schematically illustrated. For example, one or more additional operations may be performed before, after, simultaneously, or between any of the illustrated operations. Additionally, the operations may be rearranged or reordered in other embodiments. In certain circumstances, multitasking and parallel processing may be advantageous. Moreover, the separation of various system components in the embodiments described above should not be understood as requiring such separation in all embodiments, and it should be understood that the described program components and systems may generally be integrated together in a single software product or packaged into multiple software products. Additionally, other embodiments are within the scope of the following claims. In some cases, the actions recited in the claims may be performed in a different order and still achieve desirable results.

As used herein “generate” or “generating” may include specific algorithms for creating information based on or using other input information. Generating may include retrieving the input information such as from memory or as provided input parameters to the hardware performing the generating. Once obtained, the generating may include combining the input information. The combination may be performed through specific circuitry configured to provide an output indicating the result of the generating. The combination may be dynamically performed such as through dynamic selection of execution paths based on, for example, the input information, device operational characteristics (for example, hardware resources available, power level, power source, memory levels, network connectivity, bandwidth, and the like). Generating may also include storing the generated information in a memory location. The memory location may be identified as part of the request message that initiates the generating. In some implementations, the generating may return location information identifying where the generated information can be accessed. The location information may include a memory location, network locate, file system location, or the like.

Any process descriptions, elements, or blocks in the flow diagrams described herein and/or depicted in the attached figures should be understood as potentially representing modules, segments, or portions of code which include one or more executable instructions for implementing specific logical functions or steps in the process. Alternate implementations are included within the scope of the embodiments described herein in which elements or functions may be deleted, executed out of order from that shown or discussed, including substantially concurrently or in reverse order, depending on the functionality involved, as would be understood by those skilled in the art.

All of the methods and processes described above may be embodied in, and partially or fully automated via, software code modules executed by one or more general purpose computers. For example, the methods described herein may be performed by the processors 45, 52 and/or any other suitable computing device. The methods may be executed on the computing devices in response to execution of software instructions or other executable code read from a tangible computer readable medium. A tangible computer readable medium is a data storage device that can store data that is readable by a computer system. Examples of computer readable mediums include read-only memory, random-access memory, other volatile or non-volatile memory devices, CD-ROMs, magnetic tape, flash drives, and optical data storage devices.

Documents incorporated by reference in the present patent application are to be considered an integral part of the application except that, to the extent that any terms are defined in these incorporated documents in a manner that conflicts with definitions made explicitly or implicitly in the present specification, only the definitions in the present specification should be considered.

It should be emphasized that many variations and modifications may be made to the above-described embodiments, the elements of which are to be understood as being among other acceptable examples. All such modifications and variations are intended to be included herein within the scope of this disclosure. The foregoing description details certain embodiments. It will be appreciated, however, that no matter how detailed the foregoing appears in text, the systems and methods can be practiced in many ways. As it is also stated above, it should be noted that the use of particular terminology when describing certain features or aspects of the systems and methods should not be taken to imply that the terminology is being re-defined herein to be restricted to including any specific characteristics of the features or aspects of the systems and methods with which that terminology is associated. While the embodiments provide various features, examples, screen displays, user interface features, and analyses, it is recognized that other embodiments may be used.

What is claimed is:

1. A head-mounted display device (HMD) comprising:
a see-through display;
left and right video cameras disposed on a plane substantially parallel to a coronal plane and positioned symmetrically with respect to a longitudinal plane,
wherein the coronal plane and the longitudinal plane are of a user wearing the HMD, and
wherein each of the left and right video cameras is configured to simultaneously capture an image including a region of interest (ROI) within a predefined field of view (FOV);
a distance sensor configured to measure the distance from the HMD to the ROI; and
at least one processor configured to:
determine the distance from the left video camera and from the right video camera to the ROI based on the measured distance from the HMD to the ROI;
determine horizontal shift values corresponding to the determined distance from the left video camera and from the right video camera to the ROI; and
horizontally shift the display of each image of the images captured by the left and right video cameras on the see-through display by the corresponding horizontal shift value.

2. The HMD according to claim 1, wherein each of the left and right video cameras comprises a sensor.

3. The HMD according to claim 2, wherein the FOV is predefined for each of the left and right video cameras by determining a crop region on each sensor.

4. The HMD according to claim 3, wherein the crop regions of the sensors of the left and right video cameras are determined such that the left and right video cameras converge at a preselected distance from the HMD.

5. The HMD according to claim 3, wherein the crop regions of the sensors of the left and right video cameras are determined such that the images captured by the left and right video cameras at a preselected distance from the HMD fully overlap.

6. The HMD according to any one of claims 1-5, wherein the distance sensor comprises an infrared camera.

7. The HMD according to any one of claims 1-5, wherein the left and right video cameras each comprise a red-green-blue (RGB) video camera.

8. The HMD according to any one of claims 1-5, wherein the HMD is in the form of eyewear.

9. The HMD according to any one of claims 1-5, wherein the at least one processor is further configured to discard non-overlapping portions of the images.

10. The HMD according to any one of claims 1-5, wherein the at least one processor is further configured to display only the overlapping portions of the images on the see-through display.

11. The HMD according to any one of claims 1-5, wherein the at least one processor is further configured to determine focus values corresponding to the determined distances and, for each determined distance, apply the corresponding focus value to the left and right video cameras.

12. The HMD according to any one of claims 1-5, wherein the field of view includes a portion of a body of a patient.

13. The HMD according to any one of claims 1-5, wherein the at least one processor is further configured to determine a magnification value and to magnify the displayed images on the see-through display by the magnification value.

14. The HMD according to claim 13, wherein the at least one processor is further configured to overlay augmented reality images on the magnified images displayed on the see-through display.

15. The HMD according to claim 14, wherein the at least one processor is further configured to magnify the overlaid augmented reality images on the see-through display by the magnification value.

16. The HMD according to claim 14 or claim 15, wherein the augmented reality images include a 3D model of a portion of an anatomy of a patient generated from one or more pre-operative or intraoperative medical images of the portion of the anatomy of the patient.

17. The HMD according to claim 16, wherein the portion of the anatomy includes a portion of a spine of the patient.

18. A head-mounted display device (HMD) comprising:
a see-through display;
a plurality of video cameras, wherein each of the plurality of video cameras is configured to simultaneously capture an image including a region of interest (ROI) within a predefined field of view (FOV);
a distance sensor configured to measure the distance from the HMD to the ROI; and
at least one processor configured to:
determine the distance from each of the video cameras to the ROI based on the measured distance from the HMD to the ROI; and
adjust the display of each image of the images captured by the video cameras on the see-through display based on the determined distances from the video cameras to provide an improved display on the see-through display.

19. The HMD according to claim 18, wherein the plurality of video cameras comprise two video cameras positioned symmetrically about a longitudinal plane of a wearer of the head-mounted unit such that the plurality of video cameras comprise a left video camera and a right video camera.

20. The HMD according to claim 19, wherein each of the left and right video cameras comprises a sensor.

21. The HMD according to claim 20, wherein the FOV is predefined for each of the left and right video cameras by determining a crop region on each sensor.

22. The HMD according to claim 21, wherein the crop regions of the sensors of the left and right video cameras are determined such that the left and right video cameras converge at a preselected distance from the HMD.

23. The HMD according to claim 21, wherein the crop regions of the sensors of the left and right video cameras are determined such that the images captured by the left and right video cameras at a preselected distance from the HMD fully overlap.

24. The HMD according to claim 21, wherein the distance sensor comprises an infrared camera.

25. The HMD according to claim 19, wherein the left and right video cameras each comprise a red-green-blue (RGB) video camera.

26. The HMD according to any one of claims 19-25, wherein the HMD is in the form of eyewear.

27. The HMD according to any one of claims 19-25, wherein the at least one processor is further configured to discard non-overlapping portions of the images.

28. The HMD according to any one of claims 19-25, wherein the at least one processor is further configured to display only the overlapping portions of the images on the see-through display.

29. The HMD according to any one of claims 19-25, wherein the at least one processor is further configured to determine focus values corresponding to the determined distances and, for each determined distance, apply the corresponding focus value to the left and right video cameras.

30. The HMD according to any one of claims 19-25, wherein the at least one processor is further configured to determine a magnification value and to magnify the displayed images on the see-through display by the magnification value.

31. The HMD according to claim 30, wherein the at least one processor is further configured to overlay augmented reality images on the magnified images displayed on the see-through display.

32. The HMD according to claim 31, wherein the at least one processor is further configured to magnify the overlaid augmented reality images on the see-through display by the magnification value.

33. The HMD according to any one of claims 19-25, wherein the adjustment is a horizontal shift based on a horizontal shift value corresponding to the determined distances.

34. A method of providing an improved stereoscopic display on a see-through display of a head-mounted display device comprising:
simultaneously capturing images on a left and a right video camera of the head-mounted display device, the images including a region of interest (ROI) within a predefined field of view (FOV);
measuring a distance from the HMD to the ROI using a distance sensor mounted on or in the head-mounted display device; and
determining a distance from each of the left and right video cameras to the ROI based on the measured distance from the HMD to the ROI; and
adjusting the display of each image of the images captured by the left and right video cameras on the see-through display of the head-mounted display device based on the determined distances from the left and right video cameras to provide the improved stereoscopic display on the see-through display.

35. The HMD according to claim 34, wherein the see-through display comprises a left see-through display and a right see-through display.

36. The HMD according to claim 34, wherein each of the left and right video cameras comprises a sensor.

37. The HMD according to claim 34, wherein the FOV is predefined for each of the left and right video cameras by determining a crop region on each sensor.

38. The HMD according to claim 37, wherein the crop regions of the sensors of the left and right video cameras are determined such that the left and right video cameras converge at a preselected distance from the HMD.

39. The HMD according to claim 37, wherein the crop regions of the sensors of the left and right video cameras are determined such that the images captured by the left and right video cameras at a preselected distance from the HMD fully overlap.

40. The HMD according to claim 34, wherein the distance sensor comprises an infrared camera.

41. The HMD according to claim 34, wherein the left and right video cameras each comprise a red-green-blue (RGB) video camera.

42. The HMD according to any one of claims 34-41, further comprising discarding non-overlapping portions of the images.

43. The HMD according to any one of claims 34-41, further comprising displaying only the overlapping portions of the images on the see-through display.

44. The HMD according to any one of claims 34-41, further comprising determining focus values corresponding to the determined distances and, for each determined distance, applying the corresponding focus value to the left and right video cameras.

45. The HMD according to any one of claims 34-41, further comprising determining a magnification value and magnifying the displayed images on the see-through display by the magnification value.

46. The HMD according to claim 45, further comprising overlaying augmented reality images on the magnified images displayed on the see-through display.

47. The HMD according to claim 46, further comprising magnifying the overlaid augmented reality images on the see-through display by the magnification value.

48. The HMD according to any one of claims 34-41, wherein the adjustment is a horizontal shift based on a horizontal shift value corresponding to the determined distances.

49. An imaging apparatus, comprising:
a head-mounted unit, comprising a see-through display and at least one video camera, which is configured to capture images of a field of view (FOV), having a first angular extent, that is viewed through the display by a user wearing the head-mounted unit; and
a processor, which is configured to process the captured images so as to generate and present on the see-through display a magnified image of a region of interest (ROI) having a second angular extent within the FOV that is less than the first angular extent.

50. The apparatus according to claim 49, wherein the head-mounted unit comprises an eye tracker, which is configured to identify a location of a pupil of an eye of the user wearing the head-mounted unit, and wherein the processor is configured to generate the magnified image responsively to the location of the pupil.

51. The apparatus according to claim 50, wherein the eye tracker is configured to identify respective locations of pupils of both a left eye and a right eye of the user, and wherein the processor is configured to measure an interpupillary distance responsively to the identified locations of the pupils and to present respective left and right magnified images of the ROI on the see-through display responsively to the interpupillary distance.

52. The apparatus according to claim 49, wherein the magnified image presented by the processor comprises a stereoscopic image of the ROI.

53. The apparatus according to claim 52, wherein the at least one video camera comprises left and right video cameras, which are mounted respectively in proximity to left and right eyes of the user, and wherein the processor is configured to generate the stereoscopic image based on the images captured by both the left and right video cameras.

54. The apparatus according to claim 53, wherein the processor is configured to estimate a distance from the head-mounted unit to the ROI based on a disparity between the images captured by both the left and right video cameras, and to adjust the stereoscopic image responsively to the disparity.

55. The apparatus according to claim 52, wherein the see-through display comprises left and right near-eye displays, and wherein the processor is configured to generate the stereoscopic image by presenting respective left and right magnified images of the ROI on the left and right near-eye displays, while applying a horizontal shift to the left and right magnified images based on a distance from the head-mounted unit to the ROI.

56. The apparatus according to claim 55, wherein the head-mounted unit comprises a tracking system configured to measure the distance from the head-mounted unit to the ROI.

57. The apparatus according to claim 56, wherein the tracking system comprises a distance sensor.

58. The apparatus according to claim 57, wherein the distance sensor comprises an infrared camera.

59. The apparatus according to claim 56, wherein the processor is configured to measure the distance by identifying a point of contact between a tool held by the user and the ROI.

60. The apparatus according to any of claims 49 to 59, wherein the FOV comprises a part of a body of a patient undergoing a surgical procedure.

61. The apparatus according to claim 60, wherein the surgical procedure is a minimally invasive procedure.

62. The apparatus according to claim 60, wherein the surgical procedure is an open surgical procedure.

63. The apparatus according to claim 60, wherein the processor is configured to overlay an augmented reality image on the magnified image of the ROI that is presented on the see-through display.

64. A method for imaging, comprising:
capturing images of a field of view (FOV), having a first angular extent, using at least one video camera mounted on a head-mounted unit, which includes a see-through display through which a user wearing the head-mounted unit views the FOV; and
processing the captured images so as to generate and present on the see-through display a magnified image of a region of interest (ROI) having a second angular extent within the FOV that is less than the first angular extent.

65. The method according to claim 64, further comprising identifying a location of a pupil of an eye of the user wearing the head-mounted unit, wherein processing the captured images comprises generating the magnified image responsively to the location of the pupil.

66. The method according to claim 65, wherein identifying the location comprises identifying respective locations of pupils of both a left eye and a right eye of the user, and measuring an interpupillary distance responsively to the identified locations of the pupils, and wherein generating the magnified image comprises presenting respective left and right magnified images of the ROI on the see-through display responsively to the interpupillary distance.

67. The method according to claim 64, wherein the magnified image presented on the see-through display comprises a stereoscopic image of the ROI.

68. The method according to claim 67, wherein capturing the images comprises capturing left and right video images using left and right video cameras, respectively, mounted respectively in proximity to left and right eyes of the user, and wherein processing the captured images comprises generating the stereoscopic image based on the images captured by both the left and right video cameras.

69. The method according to claim 68, further comprising estimating a distance from the head-mounted unit to the ROI based on a disparity between the images captured by both the left and right video cameras, and wherein generating the stereoscopic image comprises adjusting the stereoscopic image responsively to the disparity.

70. The method according to claim 64, wherein the see-through display comprises left and right near-eye displays, and wherein generating the stereoscopic image comprises presenting respective left and right images of the ROI on the left and right near-eye displays, while applying a shift to the left and right images based on a distance from the head-mounted unit to the ROI.

71. The method according to claim 70, wherein presenting the respective left and right magnified images comprises measuring the distance from the head-mounted unit to the ROI using a tracking device on the head-mounted unit.

72. The method according to claim 71, wherein the tracking device comprises an infrared camera.

73. The method according to claim 71, wherein the tracking device comprises an optical tracker.

74. The method according to claim 71, wherein the tracking device comprises a depth sensor comprising a structured light projector.

75. The method according to claim 74, wherein measuring the distance comprises illuminating the FOV with a pattern of structured light via the structured light projector and capturing and processing an image of the pattern on the FOV.

76. The method according to claim 71, wherein presenting the respective left and right magnified images comprises measuring the distance from the head-mounted unit to the ROI by identifying a point of contact between a tool held by the user and the ROI.

77. The method according to any one of claims 64-76, wherein the FOV comprises a part of a body of a patient undergoing a surgical procedure.

78. The method according to claim 77, wherein processing the captured images comprises overlaying an augmented reality image on the magnified image of the ROI that is presented on the see-through display.

79. A computer software product, for use in conjunction with a head-mounted unit, which includes a see-through display and at least one video camera, which is configured to capture images of a field of view (FOV), having a first angular extent, that is viewed through the display by a user wearing the head-mounted unit, the product comprising a tangible, non-transitory computer-readable medium in which program instructions are stored, which instructions, when read by a processor, cause the processor to process the captured images so as to generate and present on the see-through display a magnified image of a region of interest (ROI) having a second angular extent within the FOV that is less than the first angular extent.
